(19) United States
(12) Patent Application Publication
(10) Pub. No.: US 2022/0361958 A1
(43) Pub. Date: Nov. 17, 2022

(54) REGISTRATION AND ALIGNMENT OF IMPLANTABLE SONIC WINDOWS

(71) Applicants: Kim Herbert Manwaring, phoenix, AZ (US); Jotham Manwaring, phoenix, AZ (US); Clint Folsom, phoenix, AZ (US)

(72) Inventors: Kim Herbert Manwaring, phoenix, AZ (US); Jotham Manwaring, phoenix, AZ (US); Clint Folsom, phoenix, AZ (US)

(21) Appl. No.: 17/746,239

(22) Filed: May 17, 2022

Related U.S. Application Data
(60) Provisional application No. 63/189,471, filed on May 17, 2021.

(51) Int. Cl. A61B 34/20; A61B 90/11

(52) U.S. Cl. CPC A61B 34/20; A61B 90/11; A61B 2090/103

(57) ABSTRACT

A medical device and a method of use thereof for frameless stereotaxy guided intracranial surgery. The medical device includes a central section made from a material that is transparent to ultrasound providing a sonic window, and an ultrasound reflective frame surrounding the central section. The method includes the steps of registering the ultrasound reflective frame with the frameless stereotaxy system for localization of the medical device during surgery. The medical device allows use of ultrasound imaging wherein the output of ultrasound imaging can be computationally combined with MRI or CT imaging data to compensate for anatomical changes in brain during surgery and enhanced localization and navigation to the surgery target.

### CROSS-REFERENCE TO RELATED APPLICATIONS

This application claims priority from a U.S. Provisional Patent Appl. No. 63/189,471 filed on May 17, 2021, which is incorporated herein by reference in its entirety.

### FIELD OF INVENTION

The present invention relates to a medical device and method for frameless stereotaxy, and more particularly, the present invention relates to a cranial implantable medical device that provides an ultrasound transmissive window for ultrasound imaging and a method for registration of the implanted medical device.

### BACKGROUND

Frameless stereotaxy generally refers to technologies that allow neurosurgeons to navigate to the site of surgery during intracranial surgery. Frameless stereotaxis refers broadly to the surgical and radiologic fields of image-rendered guidance of an instrument to a target in the brain, employing pre-acquired images as radiologic slices by MRI or CT scanning. The frameless stereotactic navigation devices are widely used by neurosurgeons in intracranial surgeries. For frameless stereotactic navigation, the patients undergo high-resolution MRI before surgery. Also computed tomography (CT) scan and Positron emission tomography (PET) are commonly used in combination with MRI. While MRI, CT, and PET imaging render accurately different features of brain tissue, each modality requires data acquisition prior to a surgical or medical procedure. While preparing for the surgery, these MRI scans are uploaded to the frameless stereotaxy navigation system and registration is performed after the patient's head is immobilized. Registration refers to the precise mapping of locations in the images of the dataset to the corresponding physical anatomy of the patient. Registration is normally done using skin fiducials or laser surface registration methods, both widely used. This is usually performed through the application of fiducial markers on the scalp prior to imaging and again used for registration and alignment at the surgery. Infrared reflective fiducials are common, but active markers are also employed that include LEDs and magnetic coils affixed to the scalp and/or cranium.

The known frameless stereotaxy navigation technologies have advantages; however, signification limitations are also well observed. It has been well established that pre-operative imaging sets of the brain do not necessarily and accurately render brain images with acceptable accuracy during the actual surgery on the patient. This is most commonly attributed to brain sag or deformation when the cranium has been opened, CSF has been drained, or margins of tissue have been retracted or resected. Since the registration is performed based on preoperative images, changes in the brain anatomy relative to these preoperative scans can significantly alter the accuracy of frameless stereotaxic systems.

A need is therefore appreciated for improvements in the frameless stereotactic system and procedures.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a medical device that allows using ultrasound imaging for frameless stereotaxy for near real time imaging of the brain.

It is another object of the present invention that the ultrasound imaging modality can be used in combination with MRI, CT, or PET imaging.

It is still another object of the present invention that additional fiducial markers can be provided.

It is yet another object of the present invention that the medical device allows near real time adaptations to anatomical changes to the brain during surgery.

It is a further object of the present invention that the medical device can provide access to the brain for medical instruments and endoscope.

In one aspect, disclosed is a medical device and a method of use therefor for frameless stereotaxy guided intracranial surgery. The medical device includes a central section made from a material that is transparent to ultrasound providing a sonic window, and an ultrasound reflective frame surrounding the central section. The method includes the steps of registering the ultrasound reflective frame with the frameless stereotaxy system for localization of the medical device during surgery. The medical device allows the use of ultrasound imaging wherein the output of ultrasound imaging can be computationally combined with MRI or CT imaging data to compensate, in near real time, for anatomical changes in the brain during surgery and enhanced localization and navigation to the surgery target.

In one aspect, disclosed is a method for frameless stereotaxy comprising the steps of implanting a medical device to a scalp or cranium of a patient, the medical device comprises a central section made from a material that is transparent to ultrasound providing a sonic window, and an ultrasound reflective frame surrounding the central section. The method further includes the steps of pre-operatively, marking primary fiducial markers on the scalp or cranium; obtaining an imaging dataset by one or more imaging modalities with reference to the primary fiducial markers, wherein the one or more imaging modalities is not ultrasound; feeding the imaging dataset to a frameless stereotaxy system; and upon feeding, registering and aligning the implanted medical device on the frameless stereotaxy system using a probe, wherein the probe is configured to detect the ultrasound reflective frame of the medical device. The probe is a nearfield ultrasound transducer. The one or more imaging modalities are selected from a group consisting of computed tomography, positron emission tomography, magnetic resonance imaging, and a combination thereof. The method further comprises the steps of securing the medical device using a plurality of screws, wherein the screws are configured to be visualize by the one or more imaging modalities; and upon feeding, registering the plurality of screws with the frameless stereotaxy system using a second probe. The plurality of screws is made from titanium or polyetheretherketone (PEEK).

In one implementation, the medical device has an access portal for permitting a medical instrument to pass through into the brain. The medical instrument is an endoscope configured to illuminate and visualize its path, wherein visualization data from the endoscope is fed to the frameless stereotaxy system in near real time for use in navigation.

In one aspect, disclosed is a medical device for frameless stereotaxy comprising a central section made from a material that is transparent to ultrasound providing a sonic window; and an ultrasound reflective frame surrounding the central section, wherein the medical device is configured to be implanted to a scalp or cranium, and allows for ultrasound imaging of a brain through the sonic window. The medical device further comprises an access portal, the access portal configured to permit a medical instrument to pass through into the brain.

In one aspect, the dual-purpose screws, or fasteners for securing the disclosed medical device acts as fiducial markers. The screws can incorporate MRI contrast media. Three or more fiducial markers can be employed in the sonic window for registration and localization using methods of frameless stereotaxy.

In one implementation, the highly ultrasound reflective etching on the implant is detectable as fiducial points and curves for registration and localization using methods of frameless stereotaxy.

In one implementation, the fiducial markers are etched to become infrared (IR) reflective and therefore detectable with infrared light through the scalp and the use of an infrared camera.

In one implementation, the active emitting fiducial markers can be in the form of LED's (light emitting diodes) and incorporated into the implant.

In one implementation, the active emitting fiducial markers are in the form of emitting magnetic field coils in the x, y, and z planes.

In one implementation, a plurality of fiducial markers is incorporated in the implant surface in addition to affixing screws which may also serve as fiducials.

In one implementation, the dimensions of the implantable medical device incorporating fiducial markers are of fixed measurements for calibration of the location of the implant into the cranium. The dimensions of the implantable medical device may be made unique and customized to conform to a location in the cranium and such measurements can be employed for calibration of the location of the implant

In one implementation, a registered, calibrated probe incorporates nearfield ultrasound for the detection of fiducial markers and curves in the implanted medical device. The detector in a probe of the fiducial markers or curve in the cranial implant is a capacitive or inductive electrical sensor at the tip of a calibrated probe. The detector can recognize active infrared LEDs in the medical device.

In one implementation, the precise location and orientation of an implanted medical device can be derived by transcutaneous detection of incorporated fiducial markers, boundaries, and curves in the implanted medical device.

The fiducial markers, curves, and boundaries of an implant cranial window can be employed longitudinally over time for the management of intracranial disease.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1a shows the front side of the medical device with four screws, according to an exemplary embodiment of the present invention.

FIG. 1b shows the rear side of the medical device without screws, according to an exemplary embodiment of the present invention.

FIG. 2 illustrates the medical device implanted on a skull, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be to scale.

In the following disclosure, various embodiments are described to enable the registration and alignment of an implanted medical device. It is understood that these embodiments are example implementations of the inventions and that alternative embodiments are intended to fall within the scope of this disclosure.

Disclosed are an implantable medical device and a method for registration and alignment of the implanted medical device with the frameless stereotaxy system that allows using ultrasound imaging in frameless stereotaxy systems. Ultrasound as an imaging modality cannot usefully penetrate the cranium for diagnostic or therapeutic applications. The disclosed medical device allows using ultrasound imaging across the skin to visualize the brain, just as ultrasound can visualize the brain prior to the closure of the anterior fontanel in an infant. Ultrasound imaging has been adapted to intraoperative surgical orientation to its real time component as well as its Doppler capability which can detect arterial blood flow. The brain sag or warping of the brain can be computationally adjusted to improve orientation and targeting by fusing ultrasound images with MRI or CT.

The disclosed device can be implanted on a cranium of a patient to provide an ultrasonically lucent window that allows near real time imaging of the brain using ultrasound as an imaging modality. Incorporation of fiducial markers in the implanted medical device which can be detected and imaged by MRI, CT, and PET allows alignment of rendered images with an ultrasound transducer's real time images, affording precision and safety for this form of mixed reality surgical or medical planning and intervention.

While a sonic window can allow imaging from another angle through ultrasound, it can also incorporate an access portal for guidance and labeling of a surgical target. To be sufficiently precise, however, the sonic window must be precisely localized with reference to the cranium. This can be achieved through the use of transcutaneous detected fiducials within the margins and body of the implanted medical device.

Such a registered and aligned sonic window can enable intraoperative “mixed reality” ultrasound being the real time component. Yet further, the device and method would meaningfully extend the longitudinal management of chronic neurological disease, even subsequently largely offsetting the need for irradiating CT or costly and encumbering use of MRI. Obvious disease examples include hydrocephalus controlled by shunting, brain tumor surveillance post biopsy and resection, therapeutic targeting of tissue for ablation, and even neuromodulation for seizures, movement disorders, and pain. Pathologic emotional processing conditions such as depression and obsessive-compulsive disorder may also benefit from such focused transcranial neuromodulation by ultrasound.

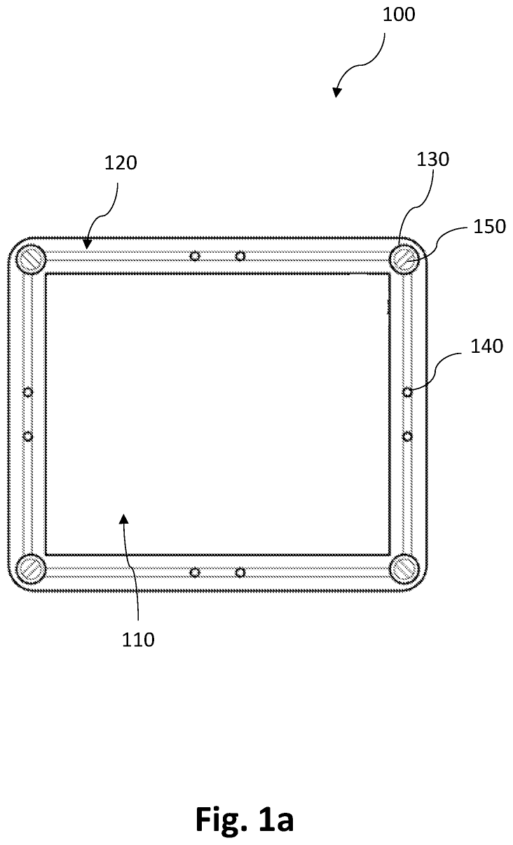

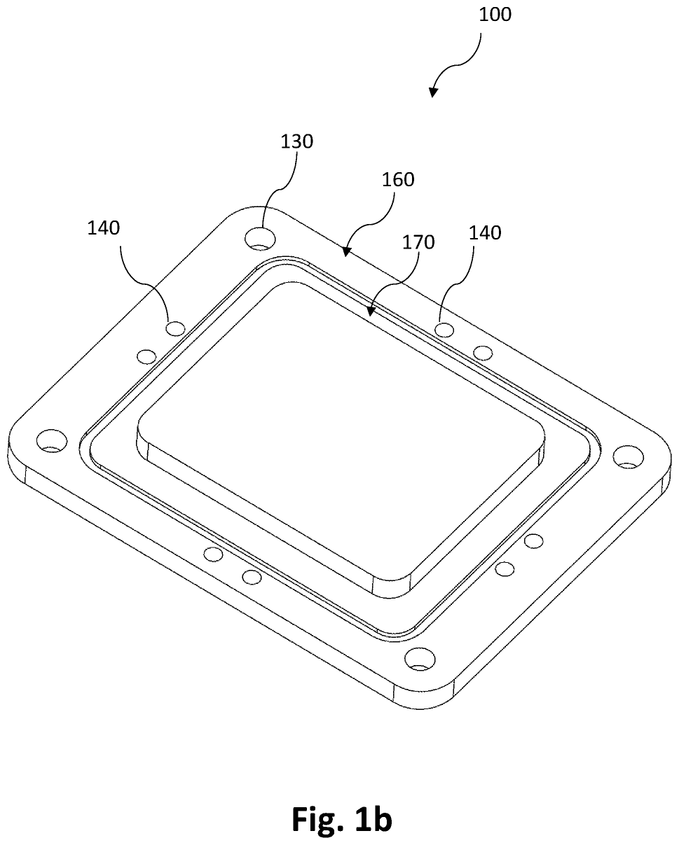

The medical device has been described in the U.S. Pat. No. 9,044,195 which is incorporated herein by reference in its entirety. FIGS. 1a and 1b show an exemplary embodiment of the medical device 100, FIG. 1a is a frontal view while FIG. 1b shows a rear view of the medical device for providing a sonic window in the cranium for ultrasound imaging. The medical device can be dimensioned to snugly adjust to the contour and align with the cranium. The medical device 100 can have a central section 110 and a frame 120 surrounding the central section. The central section can be made from a material that can transmit ultrasound in the diagnostic and therapeutic frequencies range and power ranges. In one implementation, the medical device can be fabricated using a homogeneous polymer, such as PEEK, PMMA (methyl methacrylate), or PE (polyethylene). The shape or size of the medical device varies depending on the needs of a patient and such variations are within the scope of the present invention. FIG. 1a shows a rectangular medical device 100 that has holes 130 at corners for the screws to secure the medical device to the scalp of a patient. FIG. 1a also shows the four screws 140 at four corner holes 130 of the medical device. Any other mechanism or fasteners for securing the medical device to the desired potion of the cranium are within the scope of the present invention, for example, the medical device can be secured using a press fit mechanism Also, adhesives can be used to adhere the medical device to the cranium safely and stably. In one implementation, four titanium or polyetheretherketone (PEEK) screws can be used, however, two or more such screws are within the scope of the present invention. FIG. 1a shows four pairs of additional holes 140 for the dural tack-up sutures to hold the dura snugly approximated to the implant at the surgery site. FIG. 1b shows the rear view of the medical device 100 however corner screws are not shown in FIG. 1b. The medical device can have parallel outer and inner curves as well as a thin lip that can rest on adjacent cranium and enable fixation to it. The medical device when implanted can contact the subjacent dura mater. The medical device can typically measure 2.5 by 3.5 cm and 4 mm thick and has a curvature with parallel outer and inner planes to conform to the cranium and dura. The surface of the medical device may be polished to improve ultrasound transmission without distortion. Preferably, the viewing or ultrasound transmissible zone in the medical device can be polished and consists of parallel outer 160 and inner margins 170 which diminish any ultrasound artifact or distortion, both diagnostically and therapeutically. An outer peripheral margin 160 can act as a frame around the viewing area and can be etched or roughened on at least its outer surface to allow diagnostic ultrasound across the scalp to localize the implant by strong ultrasound reflection. This outer peripheral zone can further have three or more holes for affixing the lip of the medical device as shown in FIG. 1b to the cranium. This can typically be performed with titanium screws, but other mechanisms and fasteners, such as a press fit mechanism and adhesives can also be employed. Alternatively, screws manufactured out of PEEK can be employed, with the benefit of loss of the “black spot” artifact as seen on MRI imaging.

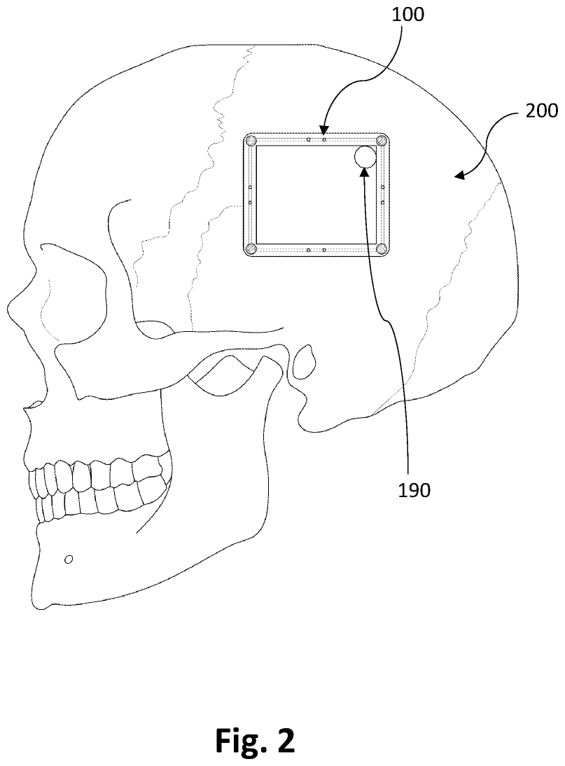

The disclosed medical device can be placed at a desired location in the cranium for providing a sonic window for both diagnostic and therapeutic ultrasound imaging. For example, the medical device can be implanted at the anterior hairline and overlying the bifrontal lobes of the brain or overlying the parietal lobe. FIG. 2 shows a view of a lateral skull 200 on which the medical device 100 is implanted overlying the parietal lobe and secured in place by four screws at the four corner holes.

The desired position for the medical device can depend upon a number of factors. For example, the medical device implanted at the at anterior hairline can allow visualization diagnostically into the brain in the traditional axial orientation of CT and MRI, but also sagittal orientation by rotating the transducer. An overlying highly ultrasound reflective border forms a picture frame around the ultrasonic viewing area and facilitates orientation to the location of the implant through an intact scalp using an ultrasound transducer. Ultrasound through the window can render typical axial images which are similar in anatomic configuration to CT and MRI axial cuts. Alternatively, the clinician can rotate the transducer to the sagittal or coronal orientations. The implanted device is typically affixed at its four corners with titanium screws which can be seen both by ultrasound nearfield methods such as 18-22 MHz as well as MRI and CT imaging in the process of data acquisition for frameless stereotaxy. Three or more such fiducials can localize computationally an implanted device of known dimensions and render its location precisely in a CT or MRI dataset. These screws can be placed into the medical device in plurality, as well, not necessarily affixing to the cranium, but serving as additional fiducial markers which can be detected by CT or MRI for purposes of improved stereotaxic localization. Yet further, the screws and medical device can be etched to become optimally reflective of infrared wavelengths which can then be visualized through the intact scalp with an infrared source and an infrared camera for localization and registration of such fiducials. Similarly, ultrasound curvilinear detection over the peripheral boundaries of the medical device can correctly locate and align the medical device.

FIG. 2 shows an access portal 190 which is a hole, typically in the upper outer corner of the medical device, the hole can be of a diameter of about 7 mm and positioned within the border region. Utilizing the co-registered ultrasound window, a surgeon can now pass an instrument percutaneously across the scalp, through the access portal, across the dura mater, and into the brain to a guided target for biopsy, delivery of medicaments, or simple aspiration. More complex yet further augmented reality can be afforded by passing an endoscope through the access portal to a target area, achieving simultaneous visual rendering of the target path and tissue destination. A calibrated instrument can be used to mark and detect fiducial markers, wherein the calibrated instrument can overlie the scalp and the implanted medical device. The tip of the calibrated instrument can detect the location of a set of fiducial markers or contours on the outer surface of the medical device, but beneath the scalp. The location and depth allow the medical device to be precisely aligned with the data set acquired through MRI or CT imaging, effectively making the medical device into an aligned imaging portal for ultrasound imaging. The detector for such implanted fiducials can include nearfield ultrasound, electrically capacitive, or inductive sensors or magnetic sensors. For example, the tip of the calibrated instrument can have an integrated capacitive sensor for the detection of a titanium screw. Alternatively, nearfield ultrasound in the 18-22 MHz range can be integrated into the tip and visualized through the scalp to see both metallic and roughened PEEK screws, as also the etched peripheral margin of the implant and its border. Four to seven fiducials are typically localized for precise alignment. Alternatively, curve or contour matching of the peripheral border or a combination with fiducial points affords precise alignment.

In certain implementations, surgery can be performed using frameless stereotaxy and the disclosed medical device implanted on a scalp of a patient at the desired position. Preoperatively, the fiducial markers can be placed on the scalp, for example using the infrared reflective method. An imaging set by MRI or CT methods can be obtained with reference to fiducial points. In the surgical suite, an infrared camera detector localizes the cranium and aligns the image set. The surgeon is thus able to employ a pointer on the scalp of the patient to localize the smallest incision and cranial opening. During surgery, the pointer can be employed to clarify anatomy as he proceeds toward the target tissue, for example, a tumor. Prior to incision, a similarly calibrated instrument with a sensor embedded in its tip can localize the implanted medical device, according to an embodiment of the present invention. For example, a nearfield ultrasound transducer can readily detect the reflection of titanium screws or the etched border zone of the medical device. A capacitive sensor in the tip of the calibrated instrument can also detect metallic fiducials. The medical device can then become a referenced and localized instrument as an implant and the images from MRI or CT can be more precisely aligned, though acquired before to surgery. When the surgeon employs diagnostic ultrasound across the ultrasonic window, enhanced accuracy of fusion of ultrasound with MRI or CT images can be rendered computationally, including a 3D representation of the trajectory and target. In an alternate implementation, a less accurate method of localization of the sonic window and access portal can be the co-registration of well-established craniofacial landmarks of the patient (medial and lateral canthi, mastoid tips, nasal tip) or by surface tracing of scalp and face with a localizing stylus.

The implanted medical device registered as described above can provide for longitudinal management and surveillance with great precision. The percutaneous passage through the access portal of the implanted medical device can be accessed by a biopsy instrument to target, for example, a recurrent tumor on the margin of a resection cavity. Simultaneous near real time ultrasound in the mixed reality environment of the clinic with the infrared guidance system can provide optimal orientation to the surgeon as well as ultrasound guided passage and biopsy. Alternatively, the calibrated and aligned sonic window enable therapeutic ultrasound to target precisely according to the MRI or CT datasets. High frequency and high intensity ultrasound can thermally ablate tissue or destroy it by cavitation. Low frequency or pulsed ultrasound can open the blood brain barrier for local delivery of intravenously administered chemotherapy. Low frequency and low intensity ultrasound can neuromodulate a seizure focus or downregulate the sensitivity of a pain threshold or movement disorder. These procedures, as a rule, are not single events, but are repeated toward a therapeutic endpoint or surveillance strategy over weeks, months, or even years of the patient's lifetime.

In certain implementations, a tip of an ultrasound emitter instrument can be registered with the frameless stereotaxy system. The implanted medical device can also be registered with the frameless stereotaxy system. Now as the clinician angles the instrument, the frameless stereotaxy system shows the target on the triaxial, and 3D display with the associated target trajectory.

In certain implementations, the medical device can include four infrared LEDs as actively emitting fiducials. IR can pass readily through the scalp and can be identified by the same IR camera array which tracks the head and various surgical tools. The LED array can be turned on by ultrasound across the scalp into a simple piezo rectifying DC circuit. Yet another active fiducial array is based on the XYZ coil which can be integrated into the medicated device and activated with transcutaneous ultrasound to a piezo in the same manner.

In certain implementations, the anchoring screws can also act as fiducials which can be imaged and registered for guidance by CT, MRI, and ultrasound. While the preferred embodiment allows the dual function of the fiducial as an anchoring screw, this is not necessary in practice. For example, a multiplicity of four to seven such fiducials could be tapped into the peripheral margin of the sonic window for purposes of registration and alignment.

For example, nearfield ultrasound in the range of 18-22 MHz can localize the tip of titanium screws as a highly reflective emitter compared to the adjacent implant material. A minimum of three, optimally four to seven such fiducials, can be placed in the curved planar implant. With its known dimension of 2.5×3.5 cm and 4 mm width with a curved surface, correct alignment with the cranium and other fiducials can be achieved. Of course, the same principle applies to custom sonic windows of different shapes, dimensions, and curves.

A tapping screw made of PEEK material can be highly dense and resistant to deformation during tapping of the cranium, if can satisfactorily fixate the medical device. It has a further advantage in that MRI dark spots characteristic of titanium are not seen. The screw tapping surface is etched to create high ultrasound reflectivity, again making localization with a nearfield 18-22 MHz instrument similarly straightforward. The pre-drilled holes in the medical device through which the screws secure the device to the cranium can be imaged by CT and MRI, just as intentional indents in implants or skull can be seen.

Screws can be made of either titanium or PEEK but having the shaft directly beneath the slotted screw surface bares a hollow or foamy zone. This region can be easily localized on MRI, just as porous PTFE (Teflon) as a product creates a surface feature. In another implementation, the hollow or foamy zone can be filled with gadolinium contrast dye or a minuscule cobalt granule with both inducing a strong MRI signal, allowing localization. Other MRI visible polymers have recently been identified for investigational application to surgical guidance and can be adapted within the scope of these inventions.

In certain implementations, the method of registering the incorporated fiducials on the medical device as described above may provide for sub-millimetric accuracy. The alternative, commonly practiced method only achieves accuracy with 2-5 mm due to known soft tissue slippage.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for frameless stereotaxy comprising the steps of:
implanting a medical device to a scalp or cranium of a patient, the medical device comprises:
a central section made from a material that is transparent to ultrasound providing a sonic window, and
an ultrasound reflective frame surrounding the central section;
pre-operatively, marking primary fiducial markers on the scalp or cranium;
obtaining an imaging dataset by one or more imaging modalities with reference to the primary fiducial markers, wherein the one or more imaging modalities is not ultrasound;
feeding the imaging dataset to a frameless stereotaxy system; and
upon feeding, registering and aligning the implanted medical device on the frameless stereotaxy system using a probe, wherein the probe is configured to detect the ultrasound reflective frame of the medical device.

2. The method according to claim 1, wherein the probe is a nearfield ultrasound transducer.

3. The method according to claim 1, wherein the one or more imaging modalities are selected from a group consisting of computed tomography, positron emission tomography, magnetic resonance imaging, and a combination thereof.

4. The method according to claim 1, wherein the method further comprises the steps of:
securing the medical device using a plurality of screws, wherein the screws are configured to be visualize by the one or more imaging modalities; and
upon feeding, registering the plurality of screws with the frameless stereotaxy system using a second probe.

5. The method according to claim 4, wherein the plurality of screws is made from titanium.

6. The method according to claim 4, wherein the plurality of screws is made from polyetheretherketone (PEEK).

7. The method according to claim 1, wherein the medical device has an access portal for permitting a medical instrument to pass through into the brain.

8. The method according to claim 7, wherein the medical instrument is an endoscope configured to illuminate and visualize its path, wherein visualization data from the endoscope is fed to the frameless stereotaxy system in near real time for use in navigation.

9. A medical device for frameless stereotaxy comprising:
a central section made from a material that is transparent to ultrasound providing a sonic window; and
an ultrasound reflective frame surrounding the central section,
wherein the medical device is configured to be implanted to a scalp or cranium, and allows for ultrasound imaging of a brain through the sonic window.

10. The medical device according to claim 9, wherein the medical device further comprises an access portal, the access portal configured to permit a medical instrument to pass through into the brain.
